(19) United States
(12) Patent Application Publication
(10) Pub. No.: US 2011/0008261 A1
(43) Pub. Date: Jan. 13, 2011

(54) METHOD TO PRODUCE HYPERPOLARISED AMINO ACIDS AND AMINOSULPHONIC ACIDS

(75) Inventors: Mathilde H. Lerche, Fredriksberg C (DK); Magnus Karlsson, Malmo (SE); Pernille R. Jensen, Kobenhavn N (DK)

Correspondence Address: GE HEALTHCARE, INC., IP DEPARTMENT 101 CARNEGIE CENTER, PRINCETON, NJ, 08540-6231, US

(73) Assignee: GE HEALTHCARE LIMITED, Buckinghamshire (GB)

(21) Appl. No.: 12/864,521

(22) PCT Filed: Feb. 3, 2009
(86) PCT No.: PCT/EP2009/051172

§ 371 Date: Sep. 28, 2010

(30) Foreign Application Priority Data
Feb. 4, 2008 (EP) 08002001.9

(51) Int. Cl. A61K 49/06; C12Q 1/02; C07C 229/26; C07C 323/58; C07D 207/10; C07C 229/06; C07C 307/02; C07C 303/34; C07C 227/14; C07C 319/12; G01R 33/44

(52) U.S. Cl. 424/9.3; 435/29; 562/563; 562/559; 562/557; 548/535; 562/575; 562/37; 324/307

(57) ABSTRACT

The invention relates to a dynamic nuclear polarisation (DNP) method for producing hyperpolarised amino acids and amino sulphonic acids and compositions for use in the method. As a sample, an ammonium salt of an amino acid, an ammonium salt of an aminosulphonic acid, a carboxylate salt of an amino acid, a sulphonate salt of an aminosulphonic acid or mixtures thereof is used.

The invention relates to a dynamic nuclear polarisation (DNP) method for producing hyperpolarised amino acids and amino sulphonic acids and compositions for use in the method.

Magnetic resonance (MR) imaging (MRI) is a technique that has become particularly attractive to physicians as images of a patients body or parts thereof can be obtained in a non-invasive way and without exposing the patient and the medical personnel to potentially harmful radiation such as X-rays. Because of its high quality images and good spatial and temporal resolution, MRI is a favourable imaging technique for imaging soft tissue and organs.

MRI may be carried out with or without MR contrast agents. However, contrast-enhanced MRI usually enables the detection of much smaller tissue changes which makes it a powerful tool for the detection of early stage tissue changes like for instance small tumours or metastases.

Several types of contrast agents have been used in MRI. Water-soluble paramagnetic metal chelates, for instance gadolinium chelates like Omniscan™ (GE Healthcare) are widely used MR contrast agents. Because of their low molecular weight they rapidly distribute into the extracellular space (i.e. the blood and the interstitium) when administered into the vasculature. They are also cleared relatively rapidly from the body.

Blood pool MR contrast agents on the other hand, for instance superparamagnetic iron oxide particles, are retained within the vasculature for a prolonged time. They have proven to be extremely useful to enhance contrast in the liver but also to detect capillary permeability abnormalities, e.g. “leaky” capillary walls in tumours which are a result of tumour angiogenesis.

Despite the undisputed excellent properties of the aforementioned contrast agents their use is not without any risks. Although paramagnetic metal chelates have usually high stability constants, it is possible that toxic metal ions are released in the body after administration. Further, these type of contrast agents show poor specificity.

WO-A-99/35508 discloses a method of MR investigation of a patient using a hyperpolarised solution of a high T₁ agent as MRI contrast agent. The term “hyperpolarisation” means enhancing the nuclear polarisation of NMR active nuclei present in the high T₁ agent, i.e. nuclei with non-zero nuclear spin, preferably ¹³C- or ¹⁵N-nuclei. Upon enhancing the nuclear polarisation of NMR active nuclei, the population difference between excited and ground nuclear spin states of these nuclei is significantly increased and thereby the MR signal intensity is amplified by a factor of hundred and more. When using a hyperpolarised ¹³C- and/or ¹⁵N-enriched high T₁ agent, there will be essentially no interference from background signals as the natural abundance of ¹³C and/or ¹⁵N is negligible and thus the image contrast will be advantageously high. The main difference between conventional MRI contrast agents and these hyperpolarised high T₁ agents is that in the former changes in contrast are caused by affecting the relaxation times of water protons in the body whereas the latter class of agents can be regarded as non-radioactive tracers, as the signal obtained arises solely from the agent.

A variety of possible high T₁ agents for use as MR imaging agents are disclosed in WO-A-99/35508, including non-endogenous and endogenous compounds. As examples of the latter intermediates in normal metabolic cycles are mentioned which are said to be preferred for imaging metabolic activity. By in vivo imaging of metabolic activity, information of the metabolic status of a tissue may be obtained and said information may for instance be used to discriminate between healthy and diseased tissue.

For instance pyruvate is a compound that plays a role in the citric acid cycle and the conversion of hyperpolarised ¹³C-pyruvate to its metabolites hyperpolarised ¹³C-lactate, hyperpolarised ¹³C-bicarbonate and hyperpolarised ¹³C-alanine can be used for in vivo MR studying of metabolic processes in the human body.

The metabolic conversion of hyperpolarised ¹³C-pyruvate to its metabolites hyperpolarised ¹³C-lactate, hyperpolarised ¹³C-bicarbonate and hyperpolarised ¹³C-alanine can be used for in vivo MR study of metabolic processes in the human body since said conversion has been found to be fast enough to allow signal detection from the parent compound, i.e. hyperpolarised ¹³C₁-pyruvate, and its metabolites. The amount of alanine, bicarbonate and lactate is dependent on the metabolic status of the tissue under investigation. The MR signal intensity of hyperpolarised ¹³C-lactate, hyperpolarised ¹³C-bicarbonate and hyperpolarised ¹³C-alanine is related to the amount of these compounds and the degree of polarisation left at the time of detection, hence by monitoring the conversion of hyperpolarised ¹³C-pyruvate to hyperpolarised ¹³C-lactate, hyperpolarised ¹³C-bicarbonate and hyperpolarised ¹³C-alanine it is possible to study metabolic processes in vivo in the human or non-human animal body by using non-invasive MR imaging and/or MR spectroscopy.

The MR signal amplitudes arising from the different pyruvate metabolites vary depending on the tissue type. The unique metabolic peak pattern formed by alanine, lactate, bicarbonate and pyruvate can be used as fingerprint for the metabolic state of the tissue under examination.

Hyperpolarised ¹³C-pyruvate may for instance be used as an MR imaging agent for assessing the viability of myocardial tissue by MR imaging as described in detail in WO-A-2006/054903 and for in vivo tumour imaging as described in detail in WO-A-2006/011810.

However, the production of hyperpolarised ¹³C-pyruvate which is suitable as an in vivo imaging agent is not without challenges. Hyperpolarised ¹³C-pyruvate is preferably obtained by dynamic nuclear polarisation (DNP) of either ¹³C-pyruvic acid or a ¹³C-pyruvate salt as described in detail in WO-A1-2006/011809, which is incorporated herein by reference.

The use of ¹³C-pyruvic acid simplifies the polarisation process since it does not crystallize upon freezing/cooling (crystallization leads to low dynamic nuclear polarisation or no polarisation at all). As a consequence no solvents and/or glass formers are needed to prepare a composition for the DNP process and thus a highly concentrated ¹³C-pyruvic acid sample can be used. However, due to its low pH a DNP agent needs to be used which is stable in the strong acid. Further, a strong base is necessary to dissolve and convert the solid hyperpolarised ¹³C-pyruvic acid after the polarisation to hyperpolarised ¹³C-pyruvate. Both the strong pyruvic acid and the strong base require careful selection of materials (e.g. dissolution medium reservoir, tubes, etc.) the compounds get in touch with.

Alternatively, a ¹³C-pyruvate salt may be used in the DNP process. Unfortunately, sodium ¹³C-pyruvate crystallizes upon freezing/cooling which makes it necessary to add glass formers. If the hyperpolarised ¹³C-pyruvate is intended to be used as in vivo imaging agent, the pyruvate concentration in the composition containing the pyruvate and glass formers is unfavourably low. Besides, the glass formers may need to be removed for in vivo use as well.

Thus preferred salts which may be used for DNP are those ¹³C-pyruvates which comprise an inorganic cation from the group consisting of NH₄⁺, K⁺, Rb⁺, Cs⁺, Ca²⁺, Sr²⁺ and Ba²⁺, preferably NH₄⁺, K⁺, Rb⁺ or Cs⁺, more preferably K⁺, Rb⁺, Cs⁺ and most preferably Cs⁺, as in detail described in PCT/NO07/00109. Most of these salts are not commercially available and need to be synthesized separately. Further, if the hyperpolarised ¹³C-pyruvate is used in vivo MR imaging it is preferred to exchange the inorganic cation from the group consisting of NH₄⁺, Rb⁺, Cs⁺, Ca²⁺, Sr²⁺ and Ba²⁺ by a physiologically very well tolerable cation like Na⁺ or meglumine. Hence an additional step is required after dissolution of the solid hyperpolarised ¹³C-pyruvate during which polarisation decays.

Other preferred salts are ¹³C-pyruvate of an organic amine or amino compound, preferably TRIS-¹³C₁-pyruvate or meglumine-¹³C₁-pyruvate, as in detail described in WO-A-2007/069909. Again these salts need to be synthesized separately.

Hence there is a need of alternative hyperpolarised imaging agents which can be used to obtain information about metabolic activity.

In protein metabolism, proteins are broken down by protease enzymes into their constituent amino acids. These amino acids are brought into the cells and can be a source of energy by being funneled into the citric acid cycle. Further, amino acids are used in several metabolic pathways in the body for the biosynthesis of other (non standard) amino acids, e.g. amino acids like citrulline in the urea cycle or other various other compounds, e.g. catecholamines from tyrosine, vitamins like niacin from tryptophan or porphyrin form glycine. Hence amino acids are important metabolic markers and hyperpolarised amino acids may be useful agents for obtaining information about metabolic activity.

We have now found a process of producing hyperpolarised amino acids by dynamic nuclear polarisation (DNP). With said process, highly concentrated samples of hyperpolarised amino acids can be obtained. This is important since a hyperpolarised amino acid which is intended to be used as agent for in vivo MR detection, e.g. MR imaging or MR spectroscopy or MR spectroscopic imaging, said amino acid needs to be administered to the patient at a high concentration, i.e. a highly concentrated sample must be used in the polarisation process. Further, the amino acids obtained by the process of the invention are highly polarised, i.e. show a high level of polarisation.

It has to be stressed that the signal of a hyperpolarised imaging agent decays due to relaxation and—upon administration to the patient's body—dilution. Hence the higher the level of polarisation the higher the MR signal which can be obtained from the agent when it has reached the target site in the patient's body.

Thus in a first aspect the invention provides a method of producing a hyperpolarised amino acid or hyperpolarised amino sulphonic acid or mixtures thereof, the method comprising
a) preparing a solution comprising a sample, a DNP agent and optionally a paramagnetic metal ion, wherein the sample is an ammonium salt of an amino acid, an ammonium salt of an aminosulphonic acid, a carboxylate salt of an amino acid, a sulphonate salt of an aminosulphonic acid or mixtures thereof;
b) freezing the solution;
c) carrying out dynamic nuclear polarisation on the frozen solution to obtain a frozen solution comprising the hyperpolarised sample; and
d) optionally liquefying and neutralizing the frozen solution obtained in step c).

The hyperpolarised amino acid and/or amino sulphonic acid obtained by the method of the invention may be used in MR-detection methods. The term “MR detection” refers to in vitro and in vivo MR detection and denotes in vitro solid state or liquid state NMR spectroscopy, MR imaging or MR spectroscopy or combined MR imaging and MR spectroscopy, i.e. MR spectroscopic imaging. The term further denotes MR spectroscopic imaging at various time points.

The terms “hyperpolarised” and “polarised” are used interchangeably hereinafter and denote a nuclear polarisation level in excess of 0.1%, more preferred in excess of 1% and most preferred in excess of 10%.

The level of polarisation may for instance be determined by solid state NMR measurements of the NMR nucleus in the frozen hyperpolarised sample. For instance, if the NMR active nucleus in the hyperpolarised sample is ¹³C, a solid state ¹³C-NMR measurement is carried out. The solid state ¹³C-NMR measurement preferably consists of a simple pulse-acquire NMR sequence using a low flip angle. The signal intensity of the hyperpolarised sample in the ¹³C-NMR spectrum is compared with signal intensity of the sample in a ¹³C-NMR spectrum acquired before the DNP polarisation process. The level of polarisation is then calculated from the ratio of the signal intensities of before and after polarisation.

In a similar way, the level of polarisation for liquid hyperpolarised samples may be determined by liquid state NMR measurements of the NMR active nucleus in the liquid hyperpolarised sample. Again the signal intensity of the liquid hyperpolarised sample is compared with the signal intensity of the liquid sample before polarisation. The level of polarisation is then calculated from the ratio of the signal intensities of before and after polarisation.

The term “sample” denotes an ammonium salt of an amino acid, an ammonium salt of an amino sulphonic acid, a carboxylate salt of an amino acid, a sulphonate salt of an aminosulphonic acid or mixtures thereof.

The term “amino acid” in the context of the invention denotes a chemical entity that comprises at least one amino group and at least one carboxy group. The at least one amino group may be a primary amino group, a secondary amino group or a tertiary amino group. An example of an amino acid according to the invention is a chemical entity that comprises one amino group and one carboxy group. In one embodiment, said one amino group and said one carboxy group are attached to the same carbon atom and examples are α-amino acids like standard or proteogenic amino acids, for instance alanine, glycine, leucine, methionine or cysteine. Both D- and L-isomers can be used in the method of the invention. Further examples of this embodiment are non-standard amino acids like sarcosine (N-methylglycine), homocysteine or betaine (trimethyl glycine). In another embodiment, said one amino group and said one carboxy group are attached to different carbon atoms and examples of this embodiment are GABA (γ-aminobutyric acid) or amino levulinic acid. In yet another embodiment, the amino acid used in the method of the invention comprises more than one amino group and/or more than one carboxy group. Examples are arginine, lysine, asparagine, ornithine, glutamine, citrulline, creatine, glutamic acid, aspartic acid or argininosuccinic acid.

The term “aminosulphonic acid” in the context of the invention denotes a chemical entity which comprises at least one amino group and at least one sulpho group, i.e. —S(O)₂OH group. The at least one amino group may be a primary amino group, a secondary amino group or a tertiary amino group. Examples of aminosulphonic acids are 1-piperidinesulphonic acid, N-(2-acetamido)-2-aminoethanesulphonic acid, 1,4-piperazine-bis-ethanesulphonic acid, 3-(N-morpholino)propanesulphonic acid, 2-(N-morpholino)ethanesulphonic acid or taurine (2-aminoethanesulphonic acid).

The terms “an ammonium salt of an amino acid” and “an ammonium salt of an aminosulphonic acid” denote a salt comprising as cation an ammonium ion of an amino acid or an ammonium ion of an aminosulphonic acid. If for instance the method of the invention is used to produce hyperpolarised alanine, in step a) a solution may be prepared which comprises an ammonium salt of alanine, wherein said ammonium salt comprises as a cation alaninium, i.e. H₃N⁺—C(CH₃)(H)—COOH. Further, if for instance the method of the invention is used to produce hyperpolarised taurine, in step a) a solution may be prepared which comprises an ammonium salt of taurine, wherein said ammonium salt comprises as a cation taurinium, i.e. H₃N⁺—CH₂—CH₂—S(O)₂—OH.

The term “a carboxylate salt of an amino acid” denotes a salt comprising as an anion the carboxylate of said amino acid. The term “a sulphonate of an aminosulphonic acid” denotes a salt comprising as an anion the sulphonate of said aminosulphonic acid. If for example the method of the invention is used to produce hyperpolarised alanine, i.e. 2-aminopropanoic acid, in step a) a solution may be prepared which comprises a carboxylate salt of alanine, wherein said carboxylate salt comprises as an anion 2-aminopropanoate. If for instance the method of the invention is used to produce hyperpolarised taurine, i.e. 2-aminoethanesulphonic acid, in step a) a solution may be prepared which comprises a sulphonate salt of taurine, wherein said sulphonate salt comprises as an anion 2-aminoethanesulphonate.

Although written in the singular form the terms “an ammonium salt of an amino acid”, “an ammonium salt of an aminosulphonic acid”, “a carboxylate salt of an amino acid” and “a sulphonate salt of an aminosulphonic acid” denote a single chemical entity or several different chemical entities. Thus a single chemical entity is for instance an ammonium salt or a carboxylate salt of a certain amino acid or an ammonium salt or a sulphonate salt of a certain aminosulphonic acid. Several different chemical entities are for instance ammonium salts or carboxylate salts of several different amino acids or ammonium salts or sulphonate salts of several different aminosulphonic acids. This is illustrated in the following paragraph with amino acids, but applies likewise to aminosulphonic acids.

Thus, as an example alanine is a certain amino acid and the method of the invention can be used to produce hyperpolarised alanine by preparing in step a) a solution comprising an ammonium salt of alanine or a carboxylate salt of alanine. Another example of a certain amino acid is GABA and the method of the invention can be used to produce hyperpolarised GABA by preparing in step a) a solution comprising an ammonium salt of GABA or a carboxylate salt of GABA. Further, as an example alanine and GABA are several different amino acids and the method of the invention can be used to produce a mixture of hyperpolarised alanine and hyperpolarised GABA by preparing in step a) a solution comprising an ammonium salt of GABA and an ammonium salt of alanine or a carboxylate salt of GABA and a carboxylate salt of alanine.

In line with the definitions provided above, the term “or mixtures thereof” denotes a mixture of an ammonium salt or a carboxylate salt of a certain amino acid or several different amino acids and an ammonium salt or sulphonate salt of a certain aminosulphonic acid or several different aminosulphonic acids. This is illustrated following paragraph.

Mixtures in the context of the invention are for instance the following:
i) a mixture of an ammonium salt of alanine and an ammonium salt of taurine
ii) a mixture of an ammonium salt of alanine and an ammonium salt of GABA and an ammonium salt of taurine and an ammonium salt of 1-piperidinesulphonic acid
iii) a mixture of a carboxylate salt of alanine and a sulphonate salt of taurine
iv) a mixture of a carboxylate salt of alanine and a carboxylate salt of GABA and a sulphonate salt of taurine and sulphonate salt of 1-piperidinesulphonic acid
v) a mixture of an ammonium salt of alanine and a sulphonate salt of taurine
vi) a mixture of a carboxylate salt of alanine and an ammonium salt of taurine
vii) a mixture of an ammonium salt of alanine and carboxylate salt of GABA and ammonium salt of taurine and sulphonate salt of 1-piperidinesulphonic acid
viii) a mixture of a carboxylate salt of alanine and ammonium salt of GABA and sulphonate salt of taurine and ammonium salt of 1-piperidinesulphonic acid

In a preferred embodiment, the method of the invention is used to produce a hyperpolarised amino acid or mixture of several hyperpolarised amino acids or a hyperpolarised aminosulphonic acid or mixtures of several hyperpolarised aminosulphonic acids.

In a more preferred embodiment the method of the invention is used to produce a hyperpolarised amino acid or a hyperpolarised aminosulphonic acid.

Preferably, the method of the invention is used to produce a hyperpolarised amino acid, more preferably a hyperpolarised α-amino acid.

The ammonium salt of an amino acid or ammonium salt of an aminosulphonic acid used in the method of the invention are either commercially available compounds, for instance many α-amino acids are commercially available as their HCl- or HBr-salts. Alternatively, ammonium salts of an amino acid or ammonium salts of an aminosulphonic acid used in the method of the invention can generally be obtained by reacting an amino acid or aminosulphonic acid with an acid. In principal any acid that has a lower pKa than the carboxyl group in the amino acid or the sulpho group in the aminosulphonic acid can be used to convert these compounds into their ammonium salts. Solubility of the ammonium salt of an amino acid or ammonium salt of an aminosulphonic acid may be hampered if the counter ion of the acid used to obtain these ammonium salts is large and/or lipophilic. Preferred acids are strong acids, more preferred strong mineral acids like hydrochloric acid (HCl), hydrobromic acid (HBr), hydroiodic acid (HI) or sulphuric acid (H₂SO₄). The most preferred acid is HCl since it is cheap and readily available. By reacting amino acids or aminosulphonic acids with HCl, ammonium chlorides are obtained which are preferably used for in vivo MR, since chlorides are well tolerated by the human or non-human animal body. However, if for any reason a less well tolerated anion is used, said anion may be exchanged after or simultaneous to step d) of the method of the invention by a physiologically well tolerated anion like chloride by methods known in the art, e.g. the use of an anion exchange column. One such reason could be that samples with higher concentration and/or higher polarisation levels can be obtained by using a specific acid for the preparation of the ammonium salt. As an example by using HI a very highly concentrated sample can be obtained but iodide is not a preferred anion when it comes to physiological tolerability. Hence said iodides may be exchanged by an anion with better physiological tolerability, e.g. chloride.

In the method of the invention, if the ammonium salt of an amino acid or ammonium salt of an aminosulphonic acid is not a commercially available compound, it may either be prepared and isolated or prepared in situ without isolating the obtained ammonium salt. The advantage of isolating the ammonium salt before preparing the solution of step a) is that the isolated salt can be characterized and it can be determined how much of the amino acid/aminosulphonic acid was actually converted into an ammonium salt. Further, if other solvents are used to prepare the solution of step a) than for the preparation of the ammonium salt, it is preferred to isolate the ammonium salt as well.

The carboxylate salts of an amino acid or sulphonate salts of an aminosulphonic acid used in the method of the invention can generally be obtained by reacting an amino acid or aminosulphonic acid with a base. In principal any base that is a stronger base than the amino group in said amino acid or aminosulphonic acid can be used to convert these compounds into their respective carboxylate and sulphonate salts. Again solubility of the carboxylate or sulphonate salts may be hampered if the counter ion of the acid used to obtain these carboxylate or sulphonate salts is large and/or lipophilic. Preferred bases are inorganic bases, more preferred aqueous solutions of alkali metal or earth alkali metal hydroxides, like aqueous solutions of sodium hydroxide (NaOH), potassium hydroxide (KOH), caesium hydroxide (CsOH), calcium hydroxide (Ca(OH)₂) or strontium hydroxide (Sr(OH)₂). The most preferred base is NaOH since it is cheap and readily available. By reacting amino acids or aminosulphonic acids with NaOH, sodium carboxylates or sodium sulphonates are obtained which are preferably used for in vivo MR, since sodium cations are very well tolerated by the human or non-human animal body. However, if for any reason a less well tolerated cation is used, said cation may be exchanged after or simultaneous to step d) of the method of the invention by a physiologically very well tolerated cation like Na⁺ or meglumine cation by methods known in the art like the use of a cation exchange column. One such reason could be that higher concentrated sample and/or polarisation levels can be obtained by using a specific base for the preparation of the carboxylate or sulphonate salt.

In the method of the invention, the carboxylate salt of an amino acid or sulphonate salt of an aminosulphonic acid may either be prepared and isolated or prepared in situ without isolating the obtained carboxylate/sulphonate salt. The advantage of isolating the salt before preparing the solution of step a) is that the isolated salt can be characterized and it can be determined how much of the amino acid/amino sulphonic acid was actually converted into the carboxylate/sulphonate salt. Further, if other solvents are used to prepare the solution of step a) than for the preparation of the carboxylate/sulphonate salt, it is preferred to isolate the carboxylate/sulphonate salt as well.

The ammonium salt of an amino acid, ammonium salt of an amino sulphonic acid, carboxylate salt of an amino acid and sulphonate salt of an amino sulphonic acid used in the method of the invention may or may not be isotopically enriched in MR active nuclei like ¹³C and/or ¹⁵N. If the hyperpolarised amino acid or aminosulphonic acid obtained by the method of the invention is used for in vivo MR, isotopic enrichment with MR active nuclei is preferred.

The ammonium salt of an amino acid, ammonium salt of an amino sulphonic acid, carboxylate salt of an amino acid and sulphonate salt of an amino sulphonic acid used in the method of the invention may be isotopically enriched in only one position of the molecule, preferably with an enrichment of at least 10%, more suitably at least 25%, more preferably at least 75% and most preferably at least 90%. Ideally, the enrichment is 100%.

Preferably, said ammonium salt of an amino acid, ammonium salt of an aminosulphonic acid, carboxylate salt of an amino acid and sulphonate salt of an aminosulphonic acid is ¹³C and/or ¹⁵N-enriched.

The optimal position for isotopic enrichment is dependent on the relaxation time of the NMR active nuclei. Preferably, ammonium salts of an amino acid, ammonium salts of an aminosulphonic acid, carboxylate salts of an amino acid and sulphonate salts of an aminosulphonic acid used in the method of the invention are isotopically enriched in positions with long T₁ relaxation time. For ¹³C-enrichment, such positions are carboxyl-C-atoms, a carbonyl-C-atoms or a quaternary C-atom with carboxyl-C-atoms being preferred. For ¹⁵N-enrichment, such positions preferably not directly proton coupled, hence tertiary amines are preferred.

Isotopic enrichment can for instance be achieved by chemical synthesis or biological labelling, both methods are known in the art and appropriate methods may be chosen depending on the specific sulphonate to be isotopically enriched.

Whether ammonium salts (in the following also referred to as acidic preparations) or carboxylates/sulphonates (in the following also referred to as basic preparations) are used in the method of the invention depends on several factors.

It is apparent that basic (acidic) preparations are the choice if the amino acid or aminosulphonic acid to be polarised does not tolerate acidic (basic) conditions, e.g. being chemically unstable under such conditions.

For α-amino acids, high relaxation rates and hence loss of polarisation was observed in solutions with a pH above 7, i.e. basic solutions. Thus, if basic preparations of α-amino acids are used for DNP, the liquefaction of the solid hyperpolarised α-amino acid needs to be carried out carefully in order to avoid loss of polarisation. This means that the basic preparation needs to be neutralized quickly after liquefaction or neutralized/liquefied simultaneously. We have however observed that basic preparations are usually easier to prepare and to handle, e.g. handling before freezing. Acidic preparations are less critical in terms of influencing the relaxation rate of the polarised α-amino acids and such acidic preparations can be pH-adjusted any time after liquefaction.

As mentioned above, the method of the invention is a method of producing a hyperpolarised amino acid or aminosulphonic acid by dynamic nuclear polarisation (DNP). In DNP, polarisation of MR active nuclei in a compound to be polarised is affected by a polarisation agent or so-called DNP agent, a compound comprising unpaired electrons. During the DNP process, energy, normally in the form of microwave radiation, is provided, which will initially excite the DNP agent. Upon decay to the ground state, there is a transfer of polarisation from the unpaired electron of the DNP agent to the NMR active nuclei of the compound to be polarised, e.g. NMR active nuclei like ¹³C and/or ¹⁵N nuclei in the sample, i.e. ammonium salt of an amino acid, ammonium salt of an amino sulphonic acid, carboxylate salt of an amino acid and sulphonate salt of an amino sulphonic acid. Generally, a moderate or high magnetic field and a very low temperature are used in the DNP process, e.g. by carrying out the DNP process in liquid helium and a magnetic field of about 1 T or above. Alternatively, a moderate magnetic field and any temperature at which sufficient polarisation enhancement is achieved may be employed. The DNP technique is for example further described in WO-A-98/58272 and in WO-A-01/96895, both of which are included by reference herein.

Generally, to polarise a chemical entity, i.e. compound, by the DNP method, a composition of the compound to be polarised and a DNP agent is prepared which is then optionally frozen and inserted into a DNP polariser (where it will freeze if it has not been frozen before) for polarisation. After the polarisation, the frozen solid hyperpolarised composition is rapidly transferred into the liquid state either by melting it or by dissolving it in a suitable dissolution medium. Dissolution is preferred and the dissolution process of a frozen hyperpolarised composition and suitable devices therefore are described in detail in WO-A-02/37132. The melting process and suitable devices for the melting are for instance described in WO-A-02/36005.

In order to obtain a high polarisation level in the compound to be polarised said compound and the DNP agent need to be in intimate contact during the DNP process. This is not the case if the composition crystallizes upon being frozen or cooled. To avoid crystallization, either glass formers need to be present in the composition or compounds need to be chosen for polarisation which do not crystallize upon being frozen but rather form a glass.

The term “glass former” in the context of this application means a chemical compound that, when added to a solution, e.g. a solution according to step a) of the method of the invention, promotes vitrification and prevents crystallization of said solution when it is cooled or frozen. Examples of preferred glass formers in the context of the invention are glycols, i.e. alcohols containing at least two hydroxyl groups, such as ethylene glycol, propylene glycol and glycerol or DMSO.

The DNP agent plays a decisive role in the DNP process as its choice has a major impact on the level of polarisation that can be achieved in the sample, i.e. amino acid or aminosulphonic acid. A variety of DNP agents—in WO-A-99/35508 denoted “OMRI contrast agents”—is known like transition metals such as chromium (V) ions, magnetic particles or organic free radicals such as nitroxide radicals or trityl radicals. The use of oxygen-based, sulphur-based or carbon-based stable trityl radicals as described in WO-A-99/35508, WO-A-88/10419, WO-A-90/00904, WO-A-91/12024, WO-A-93/02711 or WO-A-96/39367 has resulted in high levels of polarisation in a variety of different chemical entities.

In a preferred embodiment of the method of the invention, a trityl radical is used as the DNP agent. As briefly mentioned above, the large electron spin polarisation of the DNP agent, e.g. trityl radical is converted to nuclear spin polarisation of the NMR active nuclei in the sample via microwave irradiation close to the electron Larmor frequency. The microwaves stimulate communication between electron and nuclear spin systems via e-e and e-n transitions. For effective DNP, i.e. to achieve a high level of polarisation in the sample the trityl radical has to be stable and soluble in the sample or in the solution of the sample to achieve said intimate contact between the sample and the trityl radical which is necessary for the aforementioned communication between electron and nuclear spin systems.

In a preferred embodiment, the trityl radical is a radical of the formula (1)

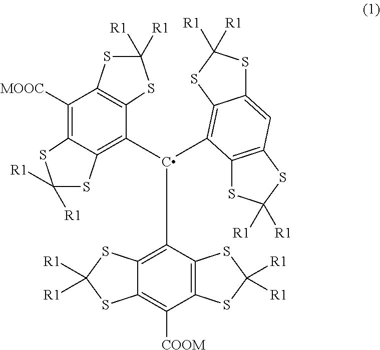

wherein
M represents hydrogen or one equivalent of a cation; and
R1 which is the same or different represents a straight chain or branched C₁-C₆-alkyl group optionally substituted by one or more hydroxyl groups or a group —(CH₂)ₙ—X—R2,
   wherein n is 1, 2 or 3;
   X is O or S; and
   R2 is a straight chain or branched C₁-C₄-alkyl group, optionally substituted by one or more hydroxyl groups.

In a preferred embodiment, M represents hydrogen or one equivalent of a physiologically tolerable cation. The term “physiologically tolerable cation” denotes a cation that is tolerated by the human or non-human animal living body. Preferably, M represents hydrogen or an alkali cation, an ammonium ion or an organic amine ion, for instance meglumine. Most preferably, M represents hydrogen or sodium.

In a further preferred embodiment, R1 is preferably the same, more preferably a straight chain or branched C₁-C₄-alkyl group, most preferably methyl, ethyl or isopropyl; or R1 is preferably the same, more preferably a straight chain or branched C₁-C₄-alkyl group which is substituted by one hydroxyl group, most preferably —CH₂—CH₂—OH; or R1 is preferably the same and represents —CH₂—OC₂H₄OH.

The aforementioned trityl radicals of formula (1) may be synthesized as described in detail in WO-A-88/10419, WO-A-90/00904, WO-A-91/12024, WO-A-93/02711, WO-A-96/39367, WO-A-97/09633, WO-A-98/39277 and WO-A-2006/011811.

In step a) of the method of the invention, a solution of the sample and the DNP agent is prepared. A solvent or a solvent mixture needs to be used to promote dissolution of the DNP agent and the sample. If the hyperpolarised amino acid or aminosulphonic acid is intended to be used as an imaging agent for in vivo MR detection, it is preferred to keep the amount of solvent to a minimum. To be used as an in vivo imaging agent, the polarised amino acid or amino sulphonic acid is usually administered in relatively high concentrations, i.e. a highly concentrated sample is preferably step c) of the method of the invention and hence the amount of solvent is preferably kept to a minimum when preparing the solution in step a). In this context, it is also important to mention that the mass of the composition containing the sample, DNP agent, solvent and optionally paramagnetic metal ion is kept as small as possible. A high mass will have a negative impact on the efficiency of the dissolution process, if dissolution is used to convert the solid composition containing the hyperpolarised sample after the DNP process into the liquid state, e.g. for using the hyperpolarised amino acid or aminosulphonic acid as an imaging agent for in vivo MR detection. This is due to the fact that for a given volume of dissolution medium in the dissolution process, the mass of the composition to dissolution medium ratio decreases, when the mass of the composition increases. Further, using certain solvents may require their removal before the hyperpolarised amino acid or aminosulphonic acid used as an MR imaging agent is administered to a human or non-human animal being since said certain solvents may not be physiologically tolerable.

If the sample used in the method of the invention is an ammonium salt of an amino acid or an ammonium salt of an amino sulphonic acid, said salt may be a commercially available salt which is dissolved in a suitable solvent, preferably water or a glass former like glycerol or glycol, or a mixture of water and a glass former. If the sample is not a commercially available salt, it is preferably prepared and isolated before being used for preparing the solution in step a). As an example the ammonium salt of ¹³C₁-alanine, i.e. alanine which is ¹³C-enriched at the carbon atom in position 1 (carboxyl carbon) may be prepared by adding an acid, for example hydrochloric acid to ¹³C₁-alanine, optionally in the presence of a solvent, for instance ethanol. The obtained ammonium salt of ¹³C₁-alanine can for example be isolated by ether precipitation and dried. The obtained ammonium salt of an amino acid or an ammonium salt of an aminosulphonic acid (e.g. ammonium salt of ¹³C₁-alanine, ¹³C₁-alaninium chloride) is then dissolved in a suitable solvent, preferably water or a glass former like glycerol or glycol, or a mixture of water and a glass former. The DNP agent, preferably a trityl radical and more preferably a trityl radical of formula (1) may either be added to the dissolved ammonium salt of an amino acid or an ammonium salt of an amino sulphonic acid as a solid or in solution. Alternatively, the DNP agent is dissolved in a suitable solvent preferably water or a glass former like glycerol or glycol, or a mixture of water and a glass former and the solid ammonium salt of an amino acid or an ammonium salt of an aminosulphonic acid is added to the dissolved DNP agent. Intimate mixing of the compounds can be promoted by several means known in the art, such as stirring, vortexing or sonication and/or gentle heating.

If the sample used in the method of the invention is a carboxylate salt of an amino acid or a sulphonate salt of an amino sulphonic acid said salt may be a commercially available salt which is dissolved in a suitable solvent, preferably water or a glass former like glycerol or glycol, or a mixture of water and a glass former. If the sample is not a commercially available salt, it is preferably prepared in situ and used in the preparation of the solution of step a) without isolating it. As an example the sodium salt of ¹³C₁-glycine, i.e. glycine which is ¹³C-enriched at the carbon atom in position 1 (carboxyl carbon) may be prepared by adding a base, for example an aqueous solution of NaOH to ¹³C₁-glycine, optionally in the presence of a solvent, for instance water. To the obtained carboxylate salt of an amino acid (e.g. sodium salt of ¹³C₁-glycine, sodium ¹³C₁-aminoethanoate) or a sulphonate salt of an aminosulphonic acid said is then added the DNP agent, preferably a trityl radical and more preferably a trityl radical of formula (1), as a solid. Alternatively, the DNP agent is dissolved in a suitable solvent preferably water or a glass former like glycerol or glycol, or a mixture of water and a glass former and the dissolved DNP agent is then added to the obtained carboxylate salt of an amino acid or a sulphonate salt of an aminosulphonic acid said. Intimate mixing of the compounds can be promoted by several means known in the art, such as stirring, vortexing or sonication and/or gentle heating.

The solution of step a) may further comprise a paramagnetic metal ion. It has been found that the presence of paramagnetic metal ions may result in increased polarisation levels in the compound to be polarised by DNP as described in detail in WO-A2-2007/064226 which is incorporated herein by reference.

The term “paramagnetic metal ion” denotes paramagnetic metal ions in the form of their salts or in chelated form, i.e. paramagnetic chelates. The latter are chemical entities comprising a chelator and a paramagnetic metal ion, wherein said paramagnetic metal ion and said chelator form a complex, i.e. a paramagnetic chelate.

In a preferred embodiment, the paramagnetic metal ion is a salt or paramagnetic chelate comprising Gd³⁺, preferably a paramagnetic chelate comprising Gd³⁺. In a more preferred embodiment, said paramagnetic metal ion is soluble and stable in the solution of step a).

As with the DNP agent described before, the sample must be in intimate contact with the paramagnetic metal ion as well. The solution comprising the sample, a DNP agent and a paramagnetic metal ion may be obtained in several ways.

In a first embodiment the sample is dissolved in a suitable solvent to obtain a solution, alternatively the sample is generated in situ in a suitable solvent as described above. To these solutions of the sample the DNP agent is added and dissolved. The DNP agent, preferably a trityl radical, might be added as a solid or in solution, e.g. dissolved in a suitable solvent, preferably water or a glass former like glycerol or glycol, or a mixture of water and a glass former. In a subsequent step, the paramagnetic metal ion is added. The paramagnetic metal ion might be added as a solid or in solution, e.g. dissolved in a suitable solvent, preferably water or a glass former like glycerol or glycol, or a mixture of water and a glass former. In another embodiment, the DNP agent and the paramagnetic metal ion are dissolved in a suitable solvent and to this solution is added the sample, either as a solid or dissolved in a suitable solvent. In yet another embodiment, the DNP agent (or the paramagnetic metal ion) is dissolved in a suitable solvent and added to the optionally dissolved sample. In a subsequent step the paramagnetic metal ion (or the DNP agent) is added to this solution, either as a solid or in solution. Preferably, the amount of solvent to dissolve the paramagnetic metal ion (or the DNP agent) is kept to a minimum. Again intimate mixing of the compounds can be promoted by several means known in the art, such as stirring, vortexing or sonication and/or gentle heating.

If a trityl radical is used as DNP agent, a suitable concentration of such a trityl radical is 1 to 25 mM, preferably 2 to 20 mM, more preferably 10 to 15 mM in the composition used for DNP. If a paramagnetic metal ion is added to the composition, a suitable concentration of such a paramagnetic metal ion is 0.1 to 6 mM (metal ion) in the composition, and a concentration of 0.3 to 4 mM is preferred.

After having prepared the solution in step a) of the method of the invention, said solution is frozen in step b). The solution can be frozen by methods known in the art, e.g. by freezing it in a freezer, in liquid nitrogen or by simply adding it to a probe-retaining cup (sample cup) and placing the sample cup in the DNP polariser, where liquid helium will freeze it. In one embodiment, the solution is frozen as “beads” before it is added to a sample cup and inserted into the polariser. Such beads may be obtained by adding the solution drop wise to liquid nitrogen. A more efficient dissolution of such beads has been observed, which is especially relevant if larger amounts of sample are polarised, for instance when the polarised amino acid or aminosulphonic acid is intended to be used in an in vivo MR detection procedure.

If a paramagnetic metal ion is present in the composition said composition may be degassed before freezing, e.g. by bubbling helium gas through the composition (e.g. for a time period of 2-15 min) but degassing can be effected by other known common methods.

As mentioned earlier, it is important that the liquefaction of basic preparations of α.-amino acids is pH controlled to avoid loss of polarisation. This may be achieved by for instance in step d) liquefying the frozen basic preparation and simultaneously neutralizing said basic preparation with the help of a dissolution medium containing an acid. Alternatively, said acid may be added to a probe-retaining cup, i.e. a cup which holds the frozen solution of step b) in the dynamic nuclear polarisation process of step c). This can be done by freezing the solution in step b) of the method of the invention in a probe-retaining cup, adding the acid on top of the frozen solution and freezing the acid. Alternatively, the acid may be frozen in a probe-retaining cup and the solution prepared in step a) of the method of the invention may be added on top of the frozen acid and then frozen in step b). This procedure results in close proximity of the acid needed for the neutralization and of the basic preparation and when liquefying the frozen solution in step d), immediate neutralization is taking place.

The DNP technique is for instance described in WO-A-98/58272 and in WO-A-01/96895, both of which are included by reference herein. Generally, a moderate or high magnetic field and a very low temperature are used in the DNP process, e.g. by carrying out the DNP process in liquid helium and a magnetic field of about 1 T or above. Alternatively, a moderate magnetic field and any temperature at which sufficient polarisation enhancement is achieved may be employed. In a preferred embodiment, the DNP process in step c) of the method of the invention is carried out in liquid helium and a magnetic field of about 1 T or above. Suitable polarisation units are for instance described in WO-A-02/37132. In a preferred embodiment, the polarisation unit comprises a cryostat and polarising means, e.g. a microwave chamber connected by a wave guide to a microwave source in a central bore surrounded by magnetic field producing means such as a superconducting magnet. The bore extends vertically down to at least the level of a region P near the superconducting magnet where the magnetic field strength is sufficiently high, e.g. between 1 and 25 T, for polarisation of the NMR active sample nuclei to take place. The bore for the probe (i.e. the frozen solution to be polarised) is preferably sealable and can be evacuated to low pressures, e.g. pressures in the order of 1 mbar or less. A probe introducing means such as a removable transporting tube can be contained inside the bore and this tube can be inserted from the top of the bore down to a position inside the microwave chamber in region P. Region P is cooled by liquid helium to a temperature low enough to for polarisation to take place, preferably temperatures of the order of 0.1 to 100 K, more preferably 0.5 to 10 K, most preferably 1 to 5 K. The probe introducing means is preferably sealable at its upper end in any suitable way to retain the partial vacuum in the bore. A probe-retaining container, such as a probe-retaining cup or sample cup, can be removably fitted inside the lower end of the probe introducing means. The probe-retaining container is preferably made of a light-weight material with a low specific heat capacity and good cryogenic properties such, e.g. KelF (polychlorotrifluoro-ethylene) or PEEK (polyetheretherketone) and it may be designed in such a way that it can hold more than one probe.

The probe is inserted into the probe-retaining container, submerged in the liquid helium and irradiated with microwaves, preferably at a frequency of about 94 GHz at 200 mW. The level of polarisation may for instance be monitored by solid state NMR measurements of the NMR active nucleus in the frozen solution comprising the hyperpolarised sample. For instance, if the NMR active nucleus in the hyperpolarised sample is ¹³C, a solid state ¹³C-NMR measurement is carried out. The solid state ¹³C-NMR measurement preferably consists of a simple pulse-acquire NMR sequence using a low flip angle. The signal intensity of the hyperpolarised sample in the ¹³C-NMR spectrum is compared with signal intensity of the sample in a ¹³C-NMR spectrum acquired before the DNP polarisation process. The level of polarisation is then calculated from the ratio of the signal intensities of before and after polarisation.

After the DNP process, the frozen solution comprising the hyperpolarised sample is optionally liquefied in step d) of the method of the invention. The term “liquefied” means transfer from a solid state to a liquid state.

If the hyperpolarised sample is used in solid state NMR spectroscopy, the optional step d) is not carried out. In solid state NMR spectroscopy the hyperpolarised solid sample may be analysed by either static or magic angle spinning solid state NMR spectroscopy.

If the hyperpolarised amino acid or aminosulphonic acid is going to be used in liquid state MR detection, step d) is carried out and liquefaction can be achieved by dissolution in an appropriate solvent or solvent mixture (dissolution medium) or by melting the solid frozen solution. Dissolution is preferred and the dissolution process and suitable devices therefore are described in detail in WO-A-02/37132. The melting process and suitable devices for the melting are for instance described in WO-A-02/36005. Briefly, a dissolution unit/melting unit is used which is either physically separated from the polariser or is a part of an apparatus that contains the polariser and the dissolution unit/melting unit. In a preferred embodiment, dissolution/melting is carried out at an elevated magnetic field, e.g. inside the polariser, to improve the relaxation and retain a maximum of the hyperpolarisation. Field nodes should be avoided and low field may lead to enhanced relaxation despite the above measures.

In order to obtain a hyperpolarised amino acid or amino sulphonic acid, the hyperpolarised sample needs to be converted to said amino acid or aminosulphonic acid. Said conversion may be carried out simultaneously or subsequently to the liquefaction, i.e. step d). Thus, in one embodiment the liquefaction is carried out by melting or dissolution and conversion is carried out after step d). In another embodiment, liquefaction and conversion are carried out simultaneously, e.g. by dissolving the frozen solution obtained in step c) in a dissolution medium which is or contains a compound that is capable of converting the hyperpolarised sample to an amino acid or amino sulphonic acid.

If the sample is an ammonium salt of an amino acid or of an amino sulphonic acid said salt can be converted to the corresponding amino acid or aminosulphonic acid by reaction (neutralization) with a base. In principal any base that is a stronger base than the amino group in said amino acid or aminosulphonic acid can be used for neutralization. Preferred bases are inorganic bases, more preferred aqueous solutions of alkali metal or earth alkali metal hydroxides, hydrogen carbonates or carbonates, like aqueous solutions of NaOH, Na₂CO₃, NaHCO₃, KOH, CsOH, Ca(OH)₂ or Sr(OH)₂. The most preferred base is NaOH since it is cheap and readily available. Further, if the hyperpolarised amino acid or aminosulphonic acid is used for in vivo MR, NaOH is preferred since the resulting sodium salts (e.g. sodium chloride) are usually well tolerated by the human or non-human animal body.

If the sample is a carboxylate salt of an amino acid or sulphonate salt of an aminosulphonic acid, said salt can be converted to the corresponding amino acid or aminosulphonic acid by reaction (neutralization) with an acid. In principal any acid that has a lower pKa than the carboxyl group in the amino acid or sulpho group in the aminosulphonic acid can be used for neutralization. Preferred acids are strong acids, even more preferred strong mineral acids like hydrochloric acid (HCl), hydrobromic acid (HBr), hydroiodic acid (HI) or sulphuric acid (H₂SO₄). The most preferred acid is HCl since it is cheap and readily available. Further, if the hyperpolarised amino acid or aminosulphonic acid is used for in vivo MR, HCl is preferred since the resulting chloride salts (e.g. sodium chloride) are usually well tolerated by the human or non-human animal body.

If the sample is a mixture of an ammonium salt and a carboxylate salt or sulphonate salt, said ammonium salt needs to be converted to the corresponding amino acid or aminosulphonic acid by reaction (neutralization) with a base and said carboxylate or sulphonate salt needs to be converted to the corresponding amino acid or aminosulphonic acid by reaction (neutralization) with an acid. Preferably, said neutralizations are carried out subsequently. If the sample comprises a carboxylate of an α-amino acid, it is preferred that neutralization with an acid takes place first, followed by neutralization of the ammonium salt present in said sample by a base.

As stated above, liquefaction in step d) is preferably carried out by dissolution with a dissolution medium that is or comprises a solvent or solvent mixture, preferably an aqueous carrier. More preferably, a physiologically tolerable and pharmaceutically accepted aqueous carrier like water or saline is used and most preferably a buffer solution, especially if the hyperpolarised amino acid or aminosulphonic acid is intended for use in an imaging medium for in vivo MR detection. For in vitro MR-detection, also non aqueous solvents or solvent mixtures may be used as or in the dissolution medium, for instance DMSO or methanol or mixtures comprising an aqueous carrier and a non aqueous solvent, for instance mixtures of DMSO and water or methanol and water. In another preferred embodiment, the dissolution medium may further comprise one or more compounds which are able to bind or complex free paramagnetic ions, e.g. chelating agents like DTPA or EDTA.

In a preferred embodiment, liquefaction in step d) is preferably carried out by dissolution with a dissolution medium, preferably a buffer solution that comprises a base or acid suitable for neutralization of the sample, i.e. converting the sample to the corresponding amino acid or aminosulphonic acid. If sample is an ammonium salt of an amino acid or of an aminosulphonic acid, and preferably if the hyperpolarised amino acid or aminosulphonic acid is intended to be used for in vivo MR detection, it is preferred to carry out step d) by using a dissolution medium comprising a buffer solution with a pH of from about 6.8 to 7 and a base. Suitable buffer solutions are for instance phosphate buffer (KH₂PO₄/Na₂HPO₄), ACES, PIPES, imidazole/HCl, BES, MOPS, HEPES, TES, TRIS, BIS-TRIS, HEPPS or TRICIN. If the sample is a carboxylate salt of an amino acid or a sulphonate salt of an amino sulphonic acid, and preferably if the hyperpolarised amino acid or aminosulphonic acid is intended to be used for in vivo MR detection, it is preferred to carry out step d) by using a dissolution medium comprising a buffer solution with a pH slightly lower than physiological pH, i.e. a pH of from about 6.8 to 7.2, and an acid. Suitable buffer solutions are for instance phosphate buffer (KH₂PO₄/Na₂HPO₄), ACES, PIPES, imidazole/HCl, BES, MOPS, HEPES, TES, TRIS, BIS-TRIS, HEPPS or TRICIN.

Subsequent to step d) of the method of the invention, the DNP agent, preferably a trityl radical, and the optional paramagnetic metal ion may be removed from the liquid containing the hyperpolarised sample or the hyperpolarised amino acid or aminosulphonic acid. Removal of these compounds is preferred if the hyperpolarised amino acid or aminosulphonic acid is intended for use in an imaging medium for in vivo MR detection. It is preferred to first convert the hyperpolarised sample to the corresponding amino acid or aminosulphonic acid and remove the DNP agent and the optional paramagnetic metal ion after said conversion has taken place.

Methods which are useful to remove the trityl radical and the paramagnetic metal ion are known in the art and described in detail in WO-A2-2007/064226 and WO-A1-2006/011809.

A liquid comprising a hyperpolarised amino acid or a hyperpolarised aminosulphonic acid or mixtures thereof produced according to the method of the invention may be used as a “conventional” MR imaging agent, i.e. providing excellent contrast enhancement for anatomical imaging in vivo, i.e. in a living human or non-human animal being. This is especially the case if the hyperpolarised amino acid or amino sulphonic acid is not metabolized or if metabolism occurs at a time scale which cannot be monitored by MR-detection.

Further, a liquid comprising a hyperpolarised amino acid or hyperpolarised aminosulphonic acid or mixtures thereof produced according to the method of the invention may be used as an imaging agent for MR detection of metabolic activity in vitro and in vivo. Amino acids can be a source of energy by being funneled into the citric acid cycle. Further, amino acids are used in several metabolic pathways in the body for the biosynthesis of other (non standard) amino acids, e.g. amino acids like citrulline in the urea cycle or other various other compounds, e.g. catecholamines from tyrosine, vitamins like niacin from tryptophan or porphyrin form glycine. Hence amino acids are important metabolic markers and hence hyperpolarised amino acids may be useful agents for obtaining information about metabolic activity by MR detection.

Another aspect of the invention is a composition comprising a sample, a DNP agent and optionally a paramagnetic metal ion, wherein the sample is an ammonium salt of an amino acid, an ammonium salt of an aminosulphonic acid, a carboxylate salt of an amino acid, a sulphonate salt of an aminosulphonic acid or mixtures thereof. In a preferred embodiment, the composition of the invention is a liquid composition which may further comprise a solvent or mixture of solvents and/or a glass former. In a preferred embodiment, the sample is an ammonium salt of an amino acid, a carboxylate salt of an amino acid or a mixture thereof.

In a further preferred embodiment, the ammonium salt of an amino acid or ammonium salt of an aminosulphonic acid is an ammonium chloride salt and/or the carboxylate salt of an amino acid or sulphonate salt of an aminosulphonic acid is a sodium carboxylate salt or sodium sulphonate salt.

In yet another preferred embodiment, the DNP agent is a trityl radical, preferably a trityl radical of formula (1). In another preferred embodiment, the composition according to the invention comprises a paramagnetic metal ion, preferably a salt or paramagnetic chelate comprising Gd³⁺.

The composition according to the invention is suitable for being used in the method of the invention, i.e. for producing a hyperpolarised amino acid or aminosulphonic acid or mixtures thereof by dynamic nuclear polarisation. Further preferred embodiments of such a composition have been discussed earlier in this application.

Yet another aspect of the invention is a composition comprising a hyperpolarised sample, a DNP agent and optionally a paramagnetic metal ion, wherein the sample is an ammonium salt of an amino acid, an ammonium salt of an aminosulphonic acid, a carboxylate salt of an amino acid, a sulphonate salt of an aminosulphonic acid or mixtures thereof. The composition according to the invention is preferably obtained by the method according to the invention.

In a preferred embodiment, the composition of the invention is a solid frozen solution which may further comprise a solvent or mixture of solvents and/or a glass former. For this preferred embodiment, the composition according to the invention is preferably obtained by the method according to the invention which comprises steps a) to c).

In a preferred embodiment, the ammonium salt of an amino acid or ammonium salt of an aminosulphonic acid is an ammonium chloride and the carboxylate salt of an amino acid or sulphonate salt of an aminosulphonic acid is a sodium carboxylate or sodium sulphonate.

In yet another preferred embodiment, the DNP agent is a trityl radical, preferably a trityl radical of formula (1). In another preferred embodiment, the composition according to the invention comprises a paramagnetic metal ion, preferably a salt or paramagnetic chelate comprising Gd³⁺.

Yet another aspect of the invention is a hyperpolarised amino acid or a hyperpolarised aminosulphonic acid or mixtures thereof. Said hyperpolarised amino acid or hyperpolarised amino sulphonic acid or mixtures thereof is preferably obtained by the method according to the invention, wherein the optional step d) is comprised in said method.

The term “amino acid” in the context of the invention denotes a chemical entity that comprises at least one amino group and at least one carboxy group. The at least one amino group may be a primary amino group, a secondary amino group or a tertiary amino group. An example of an amino acid according to the invention is a chemical entity that comprises one amino group and one carboxy group. In one embodiment, said one amino group and said one carboxy group are attached to the same carbon atom and examples are α-amino acids like standard or proteogenic amino acids, for instance alanine, glycine, leucine, methionine or cysteine. Both D- and L-isomers can be used in the method of the invention. Further examples of this embodiment are non-standard amino acids like sarcosine (N-methylglycine), homocysteine or betaine (trimethyl glycine). In another embodiment, said one amino group and said one carboxy group are attached to different carbon atoms and examples of this embodiment are GABA (γ-aminobutyric acid) or amino levulinic acid. In yet another embodiment, the amino acid used in the method of the invention comprises more than one amino group and/or more than one carboxy group. Examples are arginine, lysine, asparagine, ornithine, glutamine, citrulline, creatine, glutamic acid, aspartic acid or argininosuccinic acid.

The term “aminosulphonic acid” in the context of the invention denotes a chemical entity which comprises at least one amino group and at least one sulpho group, i.e.—S(O)₂OH group. The at least one amino group may be a primary amino group, a secondary amino group or a tertiary amino group. Examples of amino sulphonic acids are 1-piperidinesulphonic acid, N-(2-acetamido)-2-aminoethanesulphonic acid, 1,4-piperazine-bis-ethanesulphonic acid, 3-(N-morpholino)propanesulphonic acid, 2-(N-morpholino)ethanesulphonic acid or taurine (2-aminoethanesulphonic acid).

Although written in the singular form the terms “hyperpolarised amino acid” and “hyperpolarised aminosulphonic acid” denote a single hyperpolarised chemical entity or several different hyperpolarised chemical entities. Thus a single chemical entity is for instance a certain hyperpolarised amino acid like hyperpolarised glycine or like hyperpolarised alanine or hyperpolarised aminosulphonic acid like hyperpolarised taurine or like hyperpolarised N-(2-acetamido)-2-aminoethane-sulphonic acid. Several different chemical entities are for instance several different hyperpolarised amino acids like hyperpolarised glycine and hyperpolarised alanine or hyperpolarised aminosulphonic acids like hyperpolarised taurine and hyperpolarised N-(2-acetamido)-2-aminoethanesulphonic acid.

Yet another aspect of the invention is an imaging medium comprising a hyperpolarised amino acid or hyperpolarised aminosulphonic acid or mixtures thereof.

The imaging medium according to the invention may be used as imaging medium for in vitro MR detection, e.g. MR detection of cell cultures, samples, ex vivo tissue or isolated organs derived from the human or non-human animal body. For this purpose, the imaging medium is provided as a composition that is suitable for being added to, for instance, cell cultures, samples like urine, blood or saliva, ex vivo tissues like biopsy tissues or isolated organs. Such an imaging medium preferably comprises in addition to the imaging agent, i.e. the hyperpolarised amino acid or hyperpolarised amino sulphonic acid or mixtures thereof, a solvent which is compatible with and used for in vitro cell or tissue assays, for instance an aqueous carrier like water, DMSO or methanol or solvent mixtures comprising an aqueous carrier and a non aqueous solvent, for instance mixtures of DMSO and water or a buffer solution or methanol and water or a buffer solution. As it is apparent for the skilled person, pharmaceutically acceptable carriers, excipients and formulation aids may be present in such an imaging medium but are not required for such a purpose.

Further, the imaging medium according to the method of the invention may be used as imaging medium for in vivo MR detection, i.e. MR detection carried out on living human or non-human animal beings. For this purpose, the imaging medium needs to be suitable for administration to a living human or non-human animal body. Hence such an imaging medium preferably comprises in addition to the imaging agent, i.e. the hyperpolarised amino acid or hyperpolarised aminosulphonic acid or mixtures thereof, an aqueous carrier, preferably a physiologically tolerable and pharmaceutically accepted aqueous carrier like water, a buffer solution or saline. Such an imaging medium may further comprise conventional pharmaceutical or veterinary carriers or excipients, e.g. formulation aids such as stabilizers, osmolality adjusting agents, solubilising agents and the like which are conventional for diagnostic compositions in human or veterinary medicine.

### EXAMPLES

### Acidic Preparations of Amino Acids

### Example 1

### Preparation of Hyperpolarised ¹³C₁-Alanine

### Example 1a

### Preparation of an Ammonium Salt of ¹³C₁-Alanine (¹³C₁-Alaninium Chloride)

¹³C₁-alanine (100 mg, 1.1 mol, Cambridge Isotopes) was added to a 10 ml centrifugal tube, followed by addition of concentrated hydrochloric acid (145 μl, 12 M) and ethanol (1 ml, 95%). After dissolution of the ¹³C₁-alanine (sonication may be required) the resulting ammonium chloride salt of ¹³C₁-alanine (¹³C₁-alaninium chloride) was precipitated by the addition of diethyl ether (approx. 5 ml). The precipitation was collected by centrifugation and the supernatant was discarded. The precipitation was washed with diethyl ether and dried in vacuo. Recovered yield: 125 mg white powder (90%, as fine needles).

### Example 1b

### Preparation and DNP Polarisation of a Solution Comprising an ¹³C₁-Alaninium Chloride, a DNP Agent and a Paramagnetic Metal Ion

32.5 mg (0.258 mmol) of the ¹³C₁-alanine hydrochloride obtained in Example 1a was added to 42 mg of a stock solution in a micro test tube. The stock solution had been prepared by dissolving the DNP agent (trityl radical) tris(8-carboxy-2,2,6,6-(tetra(hydroxyethyl)-benzo-[1,2-4,5′]-bis-(1,3)-dithiole-4-yl)-methyl sodium salt which had been synthesised according to Example 7 of WO-A1-98/39277 and the paramagnetic metal ion (Gd-chelate of 1,3,5-tris-(N-(DO3A-acetamido)-N-methyl-4-amino-2-methylphenyl)-[1,3,5]triazinane-2,4,6-trione) which had been synthesised according to Example 4 of WO-A-2007/064226 in glycerol in such a way that a glycerol solution being 26 mM in trityl radical and 0.52 mM in Gd-chelate had been obtained. The resulting composition was sonicated to dissolve the ¹³C₁-alanine hydrochloride and produce a clear solution. The solution (65 μl, 4 M in ¹³C₁-alanine hydrochloride, 17 mM in trityl radical and 0.9 mM in Gd³⁺) was transferred with a pipette into a sample cup which was quickly lowered into liquid nitrogen to freeze the solution and then inserted into a DNP polariser. The frozen solution was polarised under DNP conditions at 1.2 K in a 3.35 T magnetic field under irradiation with microwave (93.90 GHz). Polarisation was followed by solid state ¹³C-NMR and the solid state polarisation was determined to be 40%.

### Example 1c

### Liquefaction and Neutralization

After 150 minutes of dynamic nuclear polarisation, the obtained frozen polarised solution was dissolved in a dissolution medium containing 6 ml of a phosphate buffer (20 mM, pH 6.8, 100 mg/l EDTA), aqueous NaOH (27 μl 12 M solution, 1 eq) and 30 mg NaCl. The pH of the final liquid was 6.8.

Liquid state polarisation was determined by liquid state ¹³C-NMR at 400 MHz to be 35%.

The following amino acids were polarised as acidic preparations according to Example 1

| | Sample | Solid | Concentration | Liquid |
|---|---|---|---|---|
| | concen- | state | of amino acid | state |
| | tration | polarisation | after liquefaction | polarisation |
| Amino acid | (M) | (%) | (mM) | (%) |
| ¹³C₁-glutamine | 3 | ndt | 40 | 6 |
| ¹³C₁-methionine | 3 | 41 | 40 | 26 |
| ¹³C₁-cysteine | 3 | 25 | 50 | 17 |
| ¹³C1-proline | 3 | ndt | 16 | 16 |
| ¹³C₁-glycine | 4 | 16 | 50 | 16 |

ndt = not determined.

### Basic Preparations of Amino Acids

### Example 2

### Preparation of Hyperpolarised ¹³C₁-Glutamine

### Example 2a

### Preparation and DNP Polarisation of a Solution Comprising Sodium ¹³C₁₋₂-amino-4-carbamoyl-butanoate—a Carboxylate Salt of ¹³C₁-glutamine-, a DNP Agent and a Paramagnetic Metal Ion

¹³C₁-glutamine (45.5 mg, 0.30 mmol, Cambridge Isotopes) was weighted into a micro test tube and dissolved in 23.5 μl water and 25 μl aqueous NaOH (12 M). The mixture was sonicated and gently heated to produce a clear solution. To the solution was added 5.7 mg of an aqueous solution of tris(8-carboxy-2,2,6,6-(tetra(hydroxyethyl)-benzo-[1,2-4,5′]-bis-(1,3)-dithiole-4-yl)-methyl sodium salt (trityl radical; 139 μmol/g solution) and 2.1 mg of an aqueous solution of the Gd-chelate of 1,3,5-tris-(N-(DO3A-acetamido)-N-methyl-4-amino-2-methylphenyl)-[1,3,5]triazinane-2,4,6-trione) (paramagnetic metal ion; 14.5 μmol/g solution) The resulting composition was sonicated and gently heated to produce a clear solution. The solution (approx. 75 μl, 4 M in sodium ¹³C₁-2-amino-4-carbamoyl-butanoate, 11 mM in trityl radical and 0.4 mM in Gd³⁺) was transferred with a pipette into a sample cup which was quickly lowered into liquid nitrogen to freeze the solution. The sample cup was removed from the liquid nitrogen, 25 μl aqueous HCl (12 M) were added to the sample cup. The sample cup was quickly lowered into liquid nitrogen again and then inserted into a DNP polariser. The frozen solution was polarised under DNP conditions at 1.2 K in a 3.35 T magnetic field under irradiation with microwave (93.90 GHz). Polarisation was followed by solid state ¹³C-NMR and the solid state polarisation was determined to be 35%.

### Example 2b

### Liquefaction and Neutralization

After 120 minutes of dynamic nuclear polarisation, the obtained frozen polarised solution was dissolved in a dissolution medium containing 6 ml phosphate buffer (40 mM, pH 7, 100 mg/l EDTA, 0.9% NaCl). The pH of the final solution containing the dissolved composition was 7.

Liquid state polarisation was determined by liquid state ¹³C-NMR at 400 MHz to be 30%.

The following amino acids were polarised as basic preparations according to Example 2

| | Sample | Solid | Concentration | Liquid |
|---|---|---|---|---|
| | concen- | state | of amino acid | state |
| | tration | polarisation | after liquefaction | polarisation |
| Amino acid | (M) | (%) | (mM) | (%) |
| ¹³C₁-alanine | 6 | 18 | 40 | 16 |
| ¹³C₁-leucine | 3 | 35 | 45 | 21 |
| ¹³C₁-glycine | 8 | 19 | 40 | 18 |

What is claimed is:

1. Composition comprising a sample, a DNP agent and optionally a paramagnetic metal ion, wherein the sample is an ammonium chloride salt of an amino acid, an ammonium chloride salt of an aminosulphonic acid, a sodium carboxylate salt of an amino acid, a sodium sulphonate salt of an aminosulphonic acid or mixtures thereof.

2. Composition according to claim 1 wherein the sample is an ammonium chloride salt of an amino acid or a sodium carboxylate salt of an amino acid or a mixture thereof.

3. (canceled)

4. Composition according to claim 1 wherein the sample is isotopically enriched in magnetic resonance (MR) active nuclei.

5. Composition according to claim 1 wherein the composition further comprises a solvent or mixture of solvents and/or a glass former.

6. Composition according to claim 1 wherein the DNP agent is a stable oxygen-based, sulphur-based or carbon-based trityl radical.

7. Composition according to claim 1 wherein said comprising paramagnetic metal ion is present.

8. (canceled)

9. Composition according to claim 1 wherein the sample is a hyperpolarised sample.

10. A hyperpolarised amino acid or hyperpolarised aminosulphonic acid or mixtures thereof.

11. Hyperpolarised amino acid or hyperpolarised aminosulphonic acid or mixture thereof according to claim 10 which is isotopically enriched in magnetic resonance (MR) active nuclei.

12. Hyperpolarised amino acid or hyperpolarised aminosulphonic acid or mixture thereof according to claim 10 for being obtained by dynamic nuclear polarisation.

13. Hyperpolarised amino acid or hyperpolarised aminosulphonic acid or mixture thereof according to claim 10 for use in an imaging medium for in vitro or in vivo magnetic resonance (MR) detection.

14. Imaging medium for in vitro magnetic resonance (MR) detection comprising a hyperpolarised amino acid or hyperpolarised aminosulphonic acid or mixtures thereof according to claim 10 and a solvent which is compatible with and used for in vitro cell or tissue assays.

15. Imaging medium for in vivo magnetic resonance (MR) detection comprising a hyperpolarised amino acid or hyperpolarised aminosulphonic acid or mixtures thereof according to claim 10 and an aqueous carrier.

16. Method of producing a hyperpolarised amino acid or hyperpolarised aminosulphonic acid or mixtures thereof comprising the steps of:
a) preparing a solution comprising a sample, a DNP agent and optionally a paramagnetic metal ion, wherein the sample is an ammonium salt of an amino acid, an ammonium salt of an aminosulphonic acid, a carboxylate salt of an amino acid, a sulphonate salt of an aminosulphonic acid or mixtures thereof;
b) freezing the solution;
c) carrying out dynamic nuclear polarisation on the frozen solution to obtain a frozen solution comprising the hyperpolarised sample; and
d) optionally liquefying and neutralizing the frozen solution obtained in step c).
